(19) United States
(12) Patent Application Publication
(10) Pub. No.: US 2009/0208366 A1
(43) Pub. Date: Aug. 20, 2009

(54) METHOD OF USING LIGNIN RICH FABRICS TO PREVENT MICROBIAL GROWTH IN CLEANING MATERIALS

(76) Inventor: PALLATHERI SUBRAMANIAN, Newark, DE (US)

Correspondence Address: AUSTIN BONDERER, ESQ., 644 BROOKWOOD ST., BREA, CA, 92821, US

(21) Appl. No.: 12/371,682

(22) Filed: Feb. 16, 2009

Related U.S. Application Data
(60) Provisional application No. 61/029,845, filed on Feb. 19, 2008.

(51) Int. Cl. A61L 9/00

(52) U.S. Cl. 422/5

(57) ABSTRACT

A method of preventing the creation of odor and microbial growth in fabrics used for cleaning using lignin rich fabrics.

### FIELD OF THE INVENTION

The invention relates to a new method of using lignin rich materials to prevent the formation of odor in materials exposed to conditions conducive to microbial growth.

### BACKGROUND OF THE INVENTION

There are large variety of pads and sponges that are available commercially for use as scrubbers and washers that are used widely for cleaning applications such as washing dishes, scrubbing and wiping sinks, counters, bath room walls etc. Generally these are made from fossil fuel based synthetic materials. These have some amounts of abrasive characteristics, often made by imbedding abrasive particles in a synthetic matrix as part of the article. In general, most of these develop bad smell when left under moist conditions, such as a washing sinks, wash basins, or other wet environment, due to bacterial growth. Many may contain synthetic anti-bacterial compounds incorporated into it to prevent such odor formation. Cotton fabrics are naturally derived. However, cotton is not very abrasive or stiff and is often ‘balls’ up or clumps up in use (because of its low stiffness), supporting more bacterial growth. Unless care is taken carefully to clean and dry these, these also develop bad odors when exposed to these wet environments, in a short while. There is a need for a natural based cleaning material to be able to perform satisfactorily and resist odor formation.

It has been discovered that jute resist developing an odor when used a moist environment, and it also serves as great cleaning material. The fiber is abrasive but not so much as to cause harm to delicate surfaces. Jute happens to be one of the strongest natural fibers available. It is believed that it is the lignin content and retting process that gives jute the properties of resisting development of odor.

Lignin is a complex chemical compound most commonly derived from wood and an integral part of the cell walls of plants. It is the most abundant organic polymer on Earth after cellulose, employing 30% of non-fossil organic carbon and constituting from a quarter to a third of the dry mass of wood. The compound has several unusual properties as a biopolymer, not least its heterogeneity in lacking a defined primary structure.

Lignin fills the spaces in the cell wall between cellulose, hemicellulose and pectin components, especially in tracheids, sclereids and xylem. It is covalently linked to hemicellulose and thereby crosslinks different plant polysaccharides, conferring mechanical strength to the cell wall and by extension the plant as a whole. It is particularly abundant in compression wood, but curiously scarce in tension wood.

Lignin plays a crucial part in conducting water in plant stems. The polysaccharide components of plant cell walls are highly hydrophilic and thus permeable to water, whereas lignin is more hydrophobic. The crosslinking of polysaccharides by lignin is an obstacle for water absorption to the cell wall. Thus, lignin makes it possible for the plant's vascular tissue to conduct water efficiently. Lignin is present in all vascular plants, but not in bryophytes, supporting the idea that the original function of lignin was restricted to water transport.

Lignin plays a significant role in the carbon cycle, sequestering atmospheric carbon into the living tissues of woody perennial vegetation. Lignin is one of the most slowly decomposing components of dead vegetation. Thus it retains the carbon from the atmosphere for a much longer time period and helps reduce the amount of carbon in the atmosphere.

Lignin is indigestible by mammalian and other animal enzymes, but some fungi and bacteria are able to biodegrade the polymer. The details of the reaction scheme of the biodegradation are not fully understood to date. These reactions depend on the type of wood decay—in fungi either brown rot, soft rot or white rot. The enzymes involved may employ free radicals for depolymerization reactions. Well understood lignolytic enzymes are manganese peroxidase, lignin peroxidase and cellobiose dehydrogenase. Furthermore, because of its cross-linking with the other cell wall components, it minimizes the accessibility of cellulose and hemicellulose to microbial enzymes. Hence, lignin is generally associated with reduced digestibility of the over all plant biomass, which helps defend against pathogens and pests.

A fabric that contains a good amount of lignin is jute. Jute is more commonly known in the United States as burlap. Jute is one of the strongest natural fibers available. The long staple fiber has high tensile strength and low extensibility. It also has some heat and fire resistance.

The jute fibre comes from the stem and ribbon (outer skin) of the jute plant. The fibres are first extracted by retting. The retting process consists of bundling jute stems together and immersing them in low, running water. There are two types of retting: stem and ribbon. After the retting process, stripping begins. In the stripping process, non-fibrous matter is scraped off, then the workers dig in and grab the fibres from within the jute stem.

The retting process is the process of extracting fiber from the stem or bast of the bast fiber plants. The available retting processes are: mechanical retting (hammering), chemical retting (boiling & applying chemicals), steam/vapor/dew retting, and water or microbial retting. Among them, the water or microbial retting is a century old but the most popular process in extracting fine bast fibers. It is the process of submerging plant stems, such as jute, in water, and soaking them for a period of time to loosen the fibers from the other components of the stem. Bacterial action attacks pectin and lignin, freeing the cellulose fibers. The stems are then removed and washed and subject to mechanical processing to remove the soft tissue and then dried so that all that remains are the fibers. It is believed that the pre-microbial treatment helps jute prevent the development of odor. The theory being that the microbial retting process rids the jute fabric of the compounds that would provide sustenance to microbial growth when used as a cleaning fabric.

As in typical retting, after harvesting, the jute stalks are tied into bundles and submerged in soft running water. The stalk stays submerged in water for 20 days. However, the retting process may require less time if the quality of the jute is better. In most cases, the fiber extraction process of bast fibers in water retting is done by the farmers while standing under water.

Jute is a ligno-cellulosic bast fiber. The basic constituent of jute is cellulose—the elements of which form the empirical formula (C6H10O5). Both bacteria and fungi can decompose cellulose but are dependant on moisture content. Resistance of natural fibre like jute to microbial damage may be dependant upon the physical properties (such as, crystalline, chain length, orientation of ultimate cells etc.) and the chemical constituents (such as lignin, hemicelluloses, gummy materials, and possibly, layers made from natural fibers that have antimicrobial properties etc.) The higher the lignin content the more resistant the fiber will be to rotting i.e. fungal attack. Less rotting will equate to less odor. It has also been reported that jute extracts show antimicrobial activity by Lhan et al. in Antibacterial and Antifungal Activity of Corchorus olitorus L. (Molokhia) Extracts. International journal of Natural and Engineering Sciences 1 (3): 59-61, 2007.

### DETAILED DESCRIPTION OF THE INVENTION

When experimenting with jute fabric it has been found that jute is particularly well suited to be used as a cleaning material. When used as dishwashing rag/sponge, it was found that the material did not exhibit a foul odor as is customary with ordinary sponges. Over the time in which an ordinary washcloth or a synthetic or natural sponge would develop an odor, a cleaning cloth made of jute fabric failed to exhibit such an odor.

It was noticed that the jute pad, when exposed to the wet environment, in the kitchen sink, or wash basin, without drying in between, did not develop any foul smell, like the common household sponge. This was further validated, by placing the jute pad and a general scrubbing sponge from the store, in different sealed glass jars in the presence of water, and let stand. The jute pad did not develop any bad smell even after days in this environment. The sponge developed very bad smell, presumably by bacterial and/or fungal degradation or growth. The jute pads performed in this manner naturally, without any additives or synthetic biocides. It is believed that it is the lignin content that prevents bacterial and fungal growth.

Several wipe-scrub test pads were made by sewing strips of burlap (jute fabric) about 10 cm×20 cm (4″×8″) and sewn at the edges using different sewing techniques to prevent the fibers from fraying at the sides. In some case 2 layers of Jute (burlap) were used. In several others test pads were created with one layer of jute and one layer of terry cloth were used, with the edges sewn together. Both tested types were used as dish scrubbers in the kitchen sink as well as scrubber wipes for the kitchen counter and dining table. In scrubbing, the jute surface worked remarkably well with all the dishes and counters. The softer terry side was useful in wiping and cleaning smoothly.

To test the performance of jute, materials were placed in jars with small amounts of water and the jars kept sealed with caps to maintain the humid environment. The fabrics included an all-jute fabric pad, a two layer jute/terry cloth and a common household sponge.

Next morning, the sponge had developed a real bad smell. No bad smell was in the jute or jute/cotton pad. A week later, the sponge smelled really foul. The all-jute pad did not have any bad odor other than the typical jute odor. The jute/cotton pad had a slight odor.

The pad made from Jute fabric only, was useful in scrubbing for several weeks. However its sides started to fray and the fibers started to “walk” out. This did not prevent it being useful. However, it would shorten its life and appearance. In order to extend the durability, different sewing techniques were pursued.

It was also found that a dual layer pad, where one layer was the terry cloth, did not show any signs of the jute from fraying, after several weeks of use. It appears that combining the stitching of the rigid jute fiber fabric with an over-lay of the more entangled cotton fabric supports the jute fibers from dislodging, easily.

It is maintained that use as a cleaning material is a new, never before discovered use for jute. It is also maintained that another lignin rich natural fiber, coir, will exhibit the same properties.

Obviously the shape of the lignin rich fabric can vary tremendously to meet the requirements of the desired use. Generally, a flat square or rectangular or circular or oval shapes are chosen. Decorative edges of different contours are also possible, as long as at least one side has the rough, scrubbing side. A double sided construction, where one edge is left open, makes it like a pouch or glove. One could insert one's hand/palm inside to facilitate scrubbing and cleaning. Other designs can include inserting some other innocuous fillers of similar materials such as fibers of similar fabrics, cut up or shredded fabrics or other inert fillers, such as mica or sand, saw dust etc. to make it a thicker pad. An important criteria of such fillers is that they should be made from naturally occurring plants based fibrous materials, biodegradable, or natural innocuous inorganic materials (e.g such as mica or sand) and generally not conducive to biological degradation and/or growth.

The jute fabric has also been found to be exceptional in use of cleaning windows and surfaces. When one uses this jute surface initially, to spread the cleaning fluid (like a cleaning spray) followed by wiping with the a softer, absorbing material, (that may be attached) to finish-wipe and clean, the area covered is much larger than when using a paper towel or cloth alone. The jute tends not to absorb the material and is abrasive enough to remove stubborn particular matter. However the jute does not scratch the materials being cleaned. The use of the jute makes the process more efficient, while it saves paper and cloth cleaning fabrics, it also tends to save the amount of cleaning fluid used.

Other uses envisioned in using lignin rich fabrics include bed linens, linens for children and infants. It is also envisioned that lignin rich fabrics can be incorporated in waste pads used for dogs.

While all of the experiments were conducted with Jute, it is believed that other similar high lignin containing natural fiber based (e.g. coir) products, designed in similar shapes will exhibit the same properties of inhibiting growth of organisms and are suitable for use as cleaning elements. While these fabrics are well known and have been used for years, it is maintained that this is a novel, non-obvious and never before way in which to use jute and coir.

Also, while in these examples, where jute is attached to a softer/absorbent fabric such as cotton terry, other natural fiber based soft fabrics, which have adequate resistance to bacterial growth (e.g bamboo fiber based fabric, hemp etc) be attached to complement the natural, rough fiber surface provided by jute and similar fabrics made from bast fibers such as flax, ramine, hemp, kenaf, abaca, nettles, coir, sisal as well as other natural fibers. Attached to soft fiber based fabrics such as fabrics made from

1. A method of avoiding odor in a cleaning fabric, the method comprising:
providing the fabric, wherein the cleaning fabric comprises of at least one lignin rich material;
cleaning items with the cleaning fabric; wherein when exposed the cleaning fabric is exposed to moisture, the lignin rich material resist the growth of odor producing microbes.

2. The method of claim 1, wherein the lignin rich material comprises of jute.

3. The method of claim 2, wherein the fabric further comprises of at least one other material.

4. The method of claim 3, wherein the at least one other material comprises of cotton.

5. The method of claim 1, wherein the material has at least one layer of the at least one lignin rich material and one layer of at least one other material.

6. The method of claim 1 wherein the fabric further comprises of at least one other material; the at least on lignin rich material comprises of fiber selected from the group consisting of flax, ramine, hemp, kenaf, abaca, nettles, coir, sisal; and the at least one other material comprising of a fiber from the group consisting cotton bamboo fiber, cellulosics, and natural protein fibers.

7. The method of claim 5, wherein the lignin rich materials comprises of jute and the at lease one other material comprise a natural fiber fabric, which is biodegradable and has resistance to antimicrobial growth, that is softer than jute.

8. The method of claim 5, wherein the lignin rich material comprises of jute and the at lease one other material comprises of cotton.

9. The method of claim 1, wherein the fabric comprises of two layers, wherein both layers comprise of the lignin rich material.

10. The method of claim 9, wherein the lignin rich material is jute.

11. The method of claim 1, wherein the fabric comprises a pouch

12. The method of claim 11, wherein the pouch is in the shape of a hand.
